(19) United States
(12) Patent Application Publication
(10) Pub. No.: US 2022/0401404 A1
(43) Pub. Date: Dec. 22, 2022

(54) Co-Administration Therapy to Prevent Neurodegeneration and Enhance Neuroprotection

(71) Applicants: THE REGENTS OF THE UNIVERSITY OF CALIFORNIA, Oakland, CA (US); GEORGETOWN UNIVERSITY, Washington, DC (US)

(72) Inventors: Howard J. Federoff, Irvine, CA (US); Sudhakar Raja Subramaniam, Irvine, CA (US); Massimo S. Fiandaca, Irvine, CA (US); Mark E. Mapstone, Irvine, CA (US); Xiaomin Su, Washington, DC (US)

(21) Appl. No.: 17/428,867

(22) PCT Filed: Feb. 5, 2020
(86) PCT No.: PCT/US20/16820

§ 371 Date: Aug. 5, 2021

Related U.S. Application Data
(60) Provisional application No. 62/801,271, filed on Feb. 5, 2019.

(51) Int. Cl. A61K 31/352; A61K 31/216; A61K 31/13; A61K 31/27; A61K 31/428; A61K 31/4745; A61K 36/82; A61K 36/185; A61P 25/28

(52) U.S. Cl. CPC A61K 31/352; A61K 31/216; A61K 31/13; A61K 31/27; A61K 31/428; A61K 31/4745; A61K 36/82; A61K 36/185; A61P 25/28

(57) ABSTRACT

Described herein are materials and methods for the treatment of neurodegenerative diseases by administering a combination of fenofibrate and kaempferol.

### CROSS REFERENCE TO RELATED APPLICATIONS

This present application is a U.S. national phase of International Application No. PCT/US2020/016820, filed Feb. 5, 2020, which claims the priority benefit of U.S. Provisional Patent Application No. 62/801,271, filed Feb. 5, 2019, hereby incorporated by reference in their entirety.

### STATEMENT OF GOVERNMENT SUPPORT

This invention was made with government support under W81XWH-14-1-0123 awarded by US Army Medical Research Acquisition Activity (USAMRAA). The government has certain rights in the invention.

### FIELD OF THE INVENTION

The present disclosure is directed to methods of treating a neurodegenerative disease in a subject in need thereof.

### BACKGROUND

Neurodegenerative diseases can be sporadic or familial and increase in occurrence with aging. Thus, as the average life span increases across the population, the occurrence of neurodegenerative diseases increase. As many as one of four Americans is predicted to develop a neurodegenerative condition in their lifetimes. Generally, however, the underlying mechanisms causing the conditions are not well understood and few effective treatment options are available for preventing or treating neurodegenerative diseases.

Neurodegenerative conditions feature various degrees of neuroinflammation. In addition, these disorders have been shown to include dysfunction or dysregulation of mitochondria, including that of the master mitochondrial regulator, peroxisome proliferator-activated receptor gamma (PPARγ) coactivator-1 alpha (PGC-1α). Peroxisome proliferator-activated receptor (PPAR) isoforms (e.g., α, β/δ, γ), and in particular PPARα and PPAR-γ, have been demonstrated to be neuroprotective primarily through anti-inflammatory effects, enhanced mitochondrial function, and induction of neuroprotective antioxidant genes in animal models of AD, PD, HD, and ALS, as well as in traumatic brain injury (TBI) [1-6]. PGC-1α is a transcriptional coactivator that partners with and regulates the PPARs, and induces genes involved in mitochondrial biogenesis and cellular respiration, among others[7]. These PGC-1α regulatory activities are reduced in the brains of subjects with the neurodegenerative conditions such as PD, AD and ALS [8-10].

### SUMMARY

In one aspect, described herein is a method for treating a neurodegenerative disease in a subject comprising administering fenofibrate and kaempferol to a subject in need thereof. The fenofibrate and kempferol can be administered concomitantly or sequentially.

In another aspect, described herein is a method to prevent/reduce the first-pass metabolism of fenofibrate to fenofibric acid and thereby augment levels of fenofibrate in a subject comprising administering a combination of fenofibrate and kaempferol in a molar ratio sufficient for reducing first pass metabolism of fenofibrate. In some embodiments, the levels of fenofibrate are augmented in the brain and/or visceral organs of the subject.

In some embodiments, the methods described herein further comprises administering a standard of care therapeutic to the subject. Exemplary standard of care therapeutics for the treatment of a neurodegenerative disease include, but are not limited to, the standard of care therapeutic is a dopamine precursor, dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, or tetrabenazine and derivatives thereof.

In some embodiments, the method comprises determining the subject receiving treatment has a reduced level of PGC-1α expression as compared to a control subject.

In some embodiments, the fenofibrate and kaempferol are administered at a fixed molar ratio. For example, in some embodiments, the molar ratio of fenofibrate to kaempferol is 1.2:1, 2:1, 3:1 or 4:1. In some embodiments, the molar ratio of fenofibrate to kaempferol is 3:1.

In some embodiments, administration of the fenofibrate and kempferol increases levels of fenofibrate in the brain compared to treatment with fenofibrate alone; reduces levels of oxidative stress agents in the brain or central nervous system, and/or reduces levels of inflammation in the brain or central nervous system.

In some embodiments, the subject has been diagnosed with a neurodegenerative disease. In some embodiments, the subject is at risk for developing a neurodegenerative disease. In some embodiments, the subject has an early stage neurodegenerative disease. Exemplary neurodegenerative diseases include, but are not limited to, neurodegenerative disease is Parkinson's Disease, Parkinson-plus syndrome, familial dementia, vascular dementia, Alzheimer's Disease, Huntington's Disease, multiple sclerosis, dementia with Lewy bodies, Mild Cognitive Impairment, frontotemporal dementia, retinal neurodegeneration, Amyotrophic Lateral Sclerosis (ALS) and traumatic brain injury (TBI). In some embodiments, the Parkinson-plus syndrome is multiple system atrophy (MSA), progressive supranuclear palsy (PSP) or corticobasal degeneration (CBD).

In another aspect, described herein is a method of inducing PGC-1α expression in a neural cell or a neural progenitor cell comprising contacting a neural cell or a neural progenitor cell with fenofibrate and kaempferol. In some embodiments, the contacting step is in vivo. In some embodiments, the induction of PGC-1α is PPARα independent. In some embodiments, the neural cell is a neuron (e.g., a dopaminergic neuron, or a neuron from a cortes, striatum or spinal cord of a subject). In some embodiments, the neural cell is a glial cell or astrocyte.

In any of the methods described herein, the administration of the fenofibrate and kaempferol is neuroprotective. In some embodiments, the neuroprotection comprises increasing the activity of or number of neuronal cells in the nigral region in the brain and/or reducing loss of positive terminals in the striatum.

In some embodiments, the kaempferol is from a natural source (e.g., a plant or plant extract comprising kaempferol). In some embodiments, the natural source or extract is green tea.

### BRIEF DESCRIPTION OF THE FIGURES

FIGS. 1A-1F show that fenofibrate inhibits LPS-induced inflammation in primary astrocytes derived from PGC-1α WT and PGC-1α heterozygous KO mice. Primary astrocytes derived from PGC-1α WT (PGC-1α+/+) (A-C) and PGC-1α heterozygous KO (PGC-1α+/−) (D-F) mice were treated with fenofibrate at 5, 10 and 20 μM overnight followed by LPS for 1 hour. Total RNA was isolated and IL-1β (A, D), TNF-α (B, E) and PGC-1α (C, F) gene expression were determined by RT-PCR. In PGC-1α WT primary microglia, LPS treatment increased IL-1β and TNF-α levels, and fenofibrate treatment at 20 μM significantly reduced this LPS-induced IL-1β expression (60%) (A) but failed to alter TNF-α (B) or PGC-1α (C) expression. In PGC-1α heterozygous KO primary microglia, LPS treatment increased IL-1β and TNF-α levels, and fenofibrate treatment significantly reduced this LPS-induced IL-1β expression (55%) (D) but failed to alter TNF-α (E) expression. Fenofibrate treatment at 10 and 20 μM significantly enhanced PGC-1α expression (1.5-fold) (F). ***p<0.01, LPS vs DMSO; ^{#}p<0.05, ^{##}p<0.01, ^{###}p<0.001, LPS+feno vs LPS, ANOVA with Student-Newman-Keuls post hoc analysis.

FIGS. 2A-2E. PPARα is not required for fenofibrate-mediated anti-inflammation in mouse primary astrocytes. Total RNA and protein were collected. PPARα gene expression was determined by qRT-PCR (FIG. 2A) and protein expression was determined by western blot analysis (FIG. 2B). Then 10 nM PPARα siRNA was used for the subsequent experiments. (FIGS. 2C-2E) Primary astrocytes were treated with 10 nM PPARα siRNA or scrambled siRNA for 30 hours followed by 20 μM fenofibrate for another 18 hrs. Then the cells were treated with 0.1 ng/ml LPS for 1 hr. Total RNA was extracted for PPARα (FIG. 2C), IL-1β (FIG. 2D), TNFα (FIG. 2E) gene expression. *p<0.05, **, p<0.01, One-way ANOVA followed by Bonferroni multiple comparisons test.

FIGS. 3A-3E. Fenofibrate is rapidly converted to fenofibric acid after oral administration in C57/BL naïve mice. C57/BL mice were orally administered with fenofibrate (100 mg/kg) and, brain, liver and plasma samples were collected after 2, 4, 6, 8 hours. Fenofibric acid levels in cortex (FIG. 3A), midbrain (nigra) (FIG. 3B), striatum (FIG. 3C), liver (FIG. 3D) and plasma (FIG. 3E) were determined using mass spectrometry. Fenofibric acid levels were high after 2-4 hours of fenofibrate administration in all brain tissue and plasma samples tested. Data expressed as mean±SEM.

FIG. 4. IL-1β gene expression in response to LPS insult is inhibited by fenofibrate and NOT fenofibric acid in BV2 cells. BV2 cells were incubated with fenofibric acid (FA), negative control DMSO and positive control fenofibrate (Feno) for 18 hours followed by 1 hour 0.1 ng/ml LPS treatment. Then total RNA was isolated for IL-1β gene qRT-PCR analysis. LPS exposure elevated IL-1β mRNA expression by 6-fold. Fenofibric acid treatment at 5, 10, 20 μM failed to reduce the elevated IL-1β levels but fenofibrate treatment (20 μM) significantly reduced IL-1β levels by 80%. **p<0.01, LPS+Feno vs. LPS ANOVA with Student-Newman-Keuls post hoc analysis.

FIGS. 5A-5D. Kaempferol specifically inhibits recombinant hCES1b to prevent fenofibrate hydrolysis to fenofibric acid. Different concentrations of fenofibrate were added to the assay mixture containing recombinant hCES1b (0.05 mg/mL), pre-incubated with one of the eight concentrations of kaempferol (0-50 μM) for 2 minutes in 100 mm Tris-Cl buffer (pH 7.4) at 37° C., to start the 10-minute reaction. Reaction was stopped, supernatant collected and fenofibric acid level was determined by LC-MS/MS. Ki values were calculated, and the type of inhibition was determined by fitting data to enzyme inhibition models: competitive (FIG. 5A), non-competitive (FIG. 5B), uncompetitive (FIG. 5C) and mixed (FIG. 5D) models. The samples were analyzed in duplicates and represented as mean values.

FIGS. 6A-6D. Kaempferol prevents fenofibrate hydrolysis to fenofibric acid in pooled human liver microsomes (HLM). Different concentrations of fenofibrate was added to the assay mixture containing HLM (1 mg/mL), pre-incubated with one of the eight concentrations of kaempferol (0-50 μM) for 2 minutes in 100 mm Tris-Cl buffer (pH 7.4) at 37° C., to start the 10-minute reaction. The reaction was stopped, supernatant collected and fenofibric acid level was determined by LC-MS/MS. Ki values were calculated, and the type of inhibition was determined by fitting data to enzyme inhibition models: competitive (FIG. 6A), non-competitive (FIG. 6B), uncompetitive (FIG. 6C) and mixed (FIG. 6D) models. The samples were analyzed in duplicates and represented as mean values.

FIGS. 7A and 7B. Co-delivery of fenofibrate and kaempferol (Compound X) exerted synergistic anti-inflammatory effect in BV2 cells. BV2 cells were incubated with 20 μM of fenofibrate and/or 10 or 20 μM of kaempferol for 18 hours and then exposed to 0.1 ng/ml LPS for 1 hour. Cell lysates were collected, and RNA was isolated for IL-1β (FIG. 7A) and PGC-1α (FIG. 7B) gene expression by RT-PCR. (A) LPS-exposure increased IL-1β mRNA levels (5-fold) and 20 μM fenofibrate treatment reduced this LPS-induced increase in IL-1β expression by 70%. Co-delivery of fenofibrate and kaempferol synergistically increased this anti-inflammatory effect and completely abolished LPS-induced increase in IL-1β expression. Kaempferol treatment alone (10, 20 μM) reduced LPS-induced increase in IL-1β expression by 60% (10 μM) and 85% (20 μM). (FIG. 7B) Fenofibrate treatment at 20 μM increased PGC-1α expression 2-fold. However, co-administration of fenofibrate (20 μM) and kaempferol (10, 20 μM) suppressed PGC-1α upregulation. Kaempferol treatment alone (10, 20 μM) did not enhance PGC-1α expression in BV2 cells. ***p<0.001, **p<0.01, *p<0.05 compared to DMSO control; ^{###}p<0.001, compared to LPS treatment; ^{$$$}p<0.001, compared to fenofibrate only treatment by Student's t test.

FIGS. 8A and 8B. Standard curves of hydrolysis of fenofibrate to fenofibric acid by recombinant hCES1b (FIG. 8A) and HLM (FIG. 8B). Different concentrations of fenofibrate was added to the assay mixture containing recombinant hCES1b (0.05 mg/mL) (FIG. 9A) or pooled human liver microsomes (1 mg/mL) (FIG. 9B) in 100 mm Tris-Cl buffer (pH 7.4) at 37° C. to start the 10-minute reaction. Reaction was stopped, supernatant collected and fenofibric acid level was determined by LC-MS/MS. Standard curve was plotted and the Km and Vmax values were calculated. The samples were analyzed in duplicates and represented as mean values.

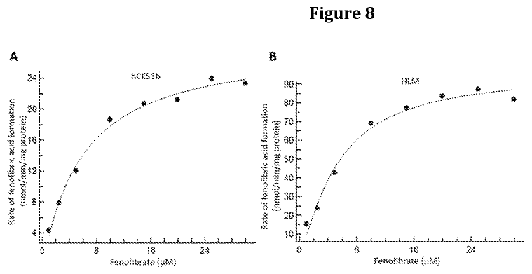

FIGS. 9A-9B. Co-delivery of kaempferol enhances brain fenofibrate levels in vivo in naïve C57/BL mice. Brain fenofibrate (FIG. 9A) and fenofibric acid (FIG. 9B) levels in mice co-administered with fenofibrate and kaempferol or fenofibrate only. Mice were pre-treated with vehicle for ‘feno only’ group or kaempferol (50 mg/kg) for ‘feno+K′ group for 2 days by oral gavage. On day 3, ‘feno only’ group mice were administered with fenofibrate (100 mg/kg) whereas ‘feno+K’ group mice were co-administered with fenofibrate (100 mg/kg) and kaempferol (50 mg/kg). Mice were sacrificed at 0, 1, 2, 4, 8, 12, 24 hours (n=4 per timepoint) after treatment and brain was collected. Brain fenofibrate and fenofibric acid levels were determined by LC-MS/MS. (FIG. 9A) Fenofibrate levels in feno+K′ group after 1 hour of oral gavage was significantly higher (˜4-fold) compared to ‘feno only’ group. ‘Feno+K’ group maintained higher levels of fenofibrate compared to ‘feno only’ group until 8 hours after oral administration. (FIG. 9B) Fenofibric acid levels in ‘feno+K’ group after 1 hour of oral gavage was significantly higher (˜2-fold) compared to ‘feno only’ group. ‘Feno+K′ group maintained higher levels of fenofibric acid compared to ‘feno only’ group until 12 hours after oral administration. Data are represented as mean±SEM. **p<0.01, *p<0.05, Student's t test compared to 0-hour timepoint.

FIGS. 10A-10I. Co-delivery of kaempferol with fenofibrate protects dopaminergic neurons in substantia nigra of mice after MPTP intoxication. C57BL mice received 5-day MPTP i.p. injection (30 mg/kg) or saline followed by 14-day i.p. drug treatment. Top panel (FIGS. 10A-10H) are the representative TH stained images of the nigral sections in the saline control, MPTP and MPTP plus fenofibrate and/or kaempferol treatment groups. Bottom panel (FIG. 10I) shows the stereological quantification of TH positive neurons in the substantia nigra. MPTP (30 mg/kg) sub-chronic treatment induced significant loss of dopaminergic neurons in substantia nigra (FIG. 10B) when compared to saline treated mice (FIG. 10A). Fenofibrate treatment (150 and 200 mg/kg) prevented MPTP-induced loss of nigral neurons (FIG. 10C, 10F). Co-administration of fenofibrate (150 mg/kg) with kaempferol (50 mg/kg) slightly increased the neuroprotective effect (FIG. 10D, 10I). Data are represented as the mean±SEM. Group A: saline+saline (n=6), Group B: MPTP+saline (n=5), Group C: MPTP+Feno150 mg/kg (n=7), Group D: MPTP+Feno150 mg/kg+K50 mg/kg (n=7), Group E: MPTP+Feno150 mg/kg+K100 mg/kg (n=7), Group F: MPTP+Feno200 mg/kg (n=5), Group G: MPTP+Feno200 mg/kg+K50 mg/kg (n=7), Group H: MPTP+Feno200 mg/kg+K100 mg/kg (n=7). ****p<0.0001, *p<0.05, unpaired Student's t test, compared to Group B: MPTP+saline.

FIGS. 11A-11I. Co-delivery of kaempferol with fenofibrate protects dopaminergic neurites in striatum of mice after MPTP intoxication. C57BL mice received 5-day MPTP i.p. injection (30 mg/kg) or saline followed by 14-day drug treatment. Top panel are the representative TH stained images of the striatal sections in the saline control, MPTP and MPTP plus fenofibrate/compound X treatment groups. Bottom panel shows TH optical density quantification in the striatum using Image J software. MPTP (30 mg/kg) sub-chronic treatment induced significant loss of striatal dopaminergic neurites (FIG. 14B) when compared to saline treated mice (FIG. 11A). Fenofibrate treatment (150 and 200 mg/kg) prevented MPTP-induced loss of striatal neurites (FIG. 11C, 11F). Co-administration of fenofibrate (150 mg/kg) with kaempferol (50 mg/kg) potentiated the neuroprotective effect (FIG. 11D, 11I). Data are represented as the mean±SEM. Group A: saline+saline (n=6), Group B: MPTP+saline (n=5), Group C: MPTP+Feno150 mg/kg (n=7), Group D: MPTP+Feno150 mg/kg+K50 mg/kg (n=7), Group E: MPTP+Feno150 mg/kg+K100 mg/kg (n=7), Group F: MPTP+Feno200 mg/kg (n=5), Group G: MPTP+Feno200 mg/kg+K50 mg/kg (n=7), Group H: MPTP+Feno200 mg/kg+K100 mg/kg (n=7). ****p<0.0001, *p,0.05, unpaired Student's t test, compared to Group B: MPTP+saline.

FIGS. 12A-12C. Green tea and capers are alternative natural sources of kaempferol and its derivatives. (FIG. 12A) TQ-MS quantification of kaempferol in different brands of caper extract and green tea extracts. Caper extracts (160-505 ng/ml/g) showed higher amounts of ‘free’ kaempferol compared to green tea extracts (14-50 ng/ml/g). QTOF qualitative analysis of kaempferol-derivatives (conjugated with complex molecules) in different brands of (FIG. 12B) caper extract and (FIG. 12C) green tea extracts. Green tea extracts (5×10⁷-1.2×10⁸ AU) showed higher amounts of kaempferol-derivatives compared to caper extract (4×10⁶-7×10⁶ AU) indicating green tea extract as a good source of compound X-derivatives. Data is expressed as mean±SEM.

### DETAILED DESCRIPTION

The present disclosure provides a method for treating neurodegenerative disease, and for inducing PGC-1α expression in a neural cell or a neural progenitor cell comprising administering a combination of fenofibrate and kaempferol in a molar ratio effective for treating neurodegenerative disease and symptoms thereof. The inventors have surprisingly found that administration of fenofibrate and kaempferol at recited molar ratios are more effective that treatment with either agent alone, and can reduce the amount of each agent required for efficacy, thus providing an unknown synergistic effect.

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

As used herein, the following terms have the meanings ascribed to them unless specified otherwise.

### Definitions

The terms “neural cells” or “population of neural cells” as used herein include both neurons (including dopaminergic neurons) and glial cells (astrocytes, oligodendrocytes, Schwann cells, and microglia). Optionally the neural cell or population of neural cells comprises central nervous system cells.

The term “neural progenitor cell” as used herein refers to a stem cell that will differentiate into a neural cell.

The term “control” is meant a value from a subject lacking the neurodegenerative disease or a known control value exemplary of a population of subjects lacking the neurodegenerative disease, or with baseline or healthy subject levels of a biomarker such as PGC1α protein. In some cases as described above, a control value can be from the same subject before the onset of a neurodegenerative disease or before the beginning of therapy therefor.

The terms “treat”, “treating”, and “treatment” refer to a method of reducing or delaying one or more effects or symptoms of a neurodegenerative disease. The subject can be diagnosed with the disease. Treatment can also refer to a method of reducing the underlying pathology rather than just the symptoms. The effect of the administration to the subject can have the effect of but is not limited to reducing one or more symptoms of the neurodegenerative disease or disorder, a reduction in the severity of the neurological disease or injury, the complete ablation of the neurological disease or injury, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

The term “prevent”, “preventing”, or “prevention” is meant a method of precluding, delaying, averting, obviating, forestalling, stopping, or hindering the onset, incidence, severity, or recurrence of the neurodegenerative disease or one or more symptoms thereof. For example, the disclosed method is considered to be a prevention if there is a reduction or delay in onset, incidence, severity, or recurrence of neurodegeneration or one or more symptoms of neurodegeneration (e.g., tremor, weakness, memory loss, rigidity, spasticity, atrophy) in a subject susceptible to neurodegeneration as compared to control subjects susceptible to neurodegeneration that did not receive fenofibrate in combination with kaempferol. The disclosed method is also considered to be a prevention if there is a reduction or delay in onset, incidence, severity, or recurrence of neurodegeneration or one or more symptoms of neurodegeneration in a subject susceptible to neurodegeneration after receiving fenofibrate or analog thereof with kaempferol as compared to the subject's progression prior to receiving treatment. Thus, the reduction or delay in onset, incidence, severity, or recurrence of neurodegeneration can be about a 10, 20, 30, 40, 50, 60, 70, 80, 90, 100%, or any amount of reduction in between.

The term “subject” as used herein means an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

The present disclosure is based on the discovery that a combination of fenofibrate or analog thereof and kaempferol at a fixed molar ratio can treat symptoms associated with a neurodegenerative disease in a subject. Fenofibrate is rapidly hydrolyzed in vivo during a first-pass through the liver, metabolized by carboxylesterase enzymes to fenofibric acid. Fenofibric acid is reported to be the active moiety that provides lipid-lowering properties of oral fenofibrate. The neuroprotective and anti-inflammatory properties of fenofibrate is attributed to fenofibrate itself, and not its metabolite fenofibric acid (see Example 6). The use of fenofibate to treat neurodegenerative diseases has been described previously in U.S. Patent Publication No. 2016/0220523, the disclosure of which is incorporated herein by reference in its entirety. The present disclosure identifies the surprising effect of the combination of fenofibrate or analog thereof and kaempferol to prevent (or reduce the rate of) the metabolism of fenofibrate into fenofibric acid, thereby augmenting levels of fenofibrate in the mouse brain (see Example 7).

In one aspect, described herein is a method of treating a neurogenerative disease in a subject comprising administering fenofibrate or analog thereof and kempferol to a subject in need thereof. The fenofibrate or analog thereof and kaempferol are preferably administered at a fixed molar ratio. In some embodiments, the molar ratio of fenofibrate or analog thereof to kaempferol is 1.5:1, 2:1, 3:1, or 4:1.

In some embodiments, the administration of fenofibrate or analog thereof and kaempferol increases levels of fenofibrate in the brain compared to treatment with fenofibrate alone; reduces levels of oxidative stress agents in the brain or central nervous system; and/or reduces levels of inflammation in the brain or central nervous system.

In some embodiments, the subject is at risk for developing a neurodegenerative disease. In some embodiments, the subject has been diagnosed with a neurodegenerative disease. One of skill in the art knows how to diagnose a subject with or at risk of developing a neurodegenerative disease. For example, one or more of the follow tests can be used: a genetic test (e.g., identification of a mutation in TDP-43 gene) or familial analysis (e.g., family history), central nervous system imaging (e.g., magnetic resonance imaging and positron emission tomography), clinical or behavioral tests (e.g., assessments of muscle weakness, tremor, muscle tone, motor skills, or memory), or laboratory tests.

The neurodegenerative disease may be an early stage neurodegenerative disease. In some embodiments, the neurodegenerative disease is Parkinson's Disease, Parkinson-plus syndrome, familial dementia, vascular dementia, Alzheimer's Disease, Huntington's Disease, multiple sclerosis, dementia with Lewy bodies, Mild Cognitive Impairment, frontotemporal dementia, retinal neurodegeneration, Amyotrophic Lateral Sclerosis (ALS) or traumatic brain injury (TBI). In some embodiments, Parkinson-plus syndrome is multiple system atrophy (MSA), progressive supranuclear palsy (PSP) or corticobasal degeneration (CBD).

Also described herein is a method to prevent/reduce the first-pass metabolism of fenofibrate to fenofibric acid comprising administering fenofibrate or analog thereof and kaempferol in a molar ratio sufficient to reduce first-pass metabolism of fenofibrate.

In another aspect, described herein is a method of inducing PGC-1α expression in a neural cell or neural progenitor cells comprising contacting the cell with fenofibrate or analog thereof or kaempferol. The contacting step can be performed either in vivo or in vitro. In some embodiments, the neural cell is a neuron. In some embodiments, the neuron is a dopaminergic neuron. In some embodiments, the neuron is a neuron in the cortex, striatum or spinal cord of a subject. In some embodiments, the neural cell is a glial cell or astrocyte.

Neurodegenerative Diseases

In some embodiments, the methods described herein comprise administering the fenofibrate and kaempferol to a subject that has been diagnosed with a neurodegenerative disease. In some embodiments, the methods described herein comprise administering the fenofibrate and kaempferol to a subject that is at risk for developing a neurodegenerative disease. In some embodiments, the subject has an early stage neurodegenerative disease.

Exemplary neurodegenerative diseases include, but are not limited to, Parkinson's Disease, Parkinson-plus syndrome, familial dementia, vascular dementia, Alzheimer's Disease, Huntington's Disease, multiple sclerosis, dementia with Lewy bodies, Mild Cognitive Impairment, frontotemporal dementia, retinal neurodegeneration, Amyotrophic Lateral Sclerosis (ALS) and traumatic brain injury (TBI). In some embodiments, the Parkinson-plus syndrome is multiple system atrophy (MSA), progressive supranuclear palsy (PSP) or corticobasal degeneration (CBD).

Alzheimer's disease (AD) is characterized by chronic, progressive neurodegeneration. Neurodegeneration in AD involves early synaptotoxicity, neurotransmitter disturbances, accumulation of extracellular β-amyloid (Aβ) deposits and intracellular neurofibrils, and gliosis and at later stages loss of neurons and associated brain atrophy (Danysz et al., Br J Pharmacol. 167:324-352, 2012). Early studies indicated Aβ peptides may have the ability to enhance glutamate toxicity in human cerebral cortical cell cultures (Mattson et al., J Neurosci. 12:376-389, 1992; Li et al., J Neurosci. 31(18):6627-38, 2011).

In some embodiments, the subject has preclinical or incipient Alzheimer's Disease. The term “incipient Alzheimer's disease,” as used herein, refers to stages of Alzheimer's disease that are less severe and/or have an earlier onset than mild to moderate disease. The term “incipient Alzheimer's disease” includes predementia (also known as, and referred to herein as, prodromal) disease as well as preclinical disease (which includes asymptomatic as well as presymptomatic disease). The diagnostic criteria used to assess what type of Alzheimer's disease a patent has can be determined using the criteria published in The Lancet Neurology, 2007, Volume 6, Issue 8, pages 734-746; and The Lancet Neurology, 2010, Volume 9, Issue 11, pages 1118-1127, the disclosures of which are incorporated herein by reference in their entireties.

It is contemplated herein that administration of a fenofibrate or analog thereof and kaempferol as described herein in combination alleviates or treat one or more symptoms associated with a neurodegenerative disease. Such symptoms, include but are not limited to, one or more motor skills, cognitive function, dystonia, chorea, psychiatric symptoms such as depression, brain and striatal atrophies, and neuronal dysfunction.

It is contemplated that the administration results in a slower progression of total motor score compared to a subject not receiving treatment as described herein. In some embodiments, the slower progression is a result in improvement in one or more motor scores selected from the group consisting of chorea subscore, balance and gait subscore, hand movements subscore, eye movement subscore, maximal dystonia subscore and bradykinesia assessment.

Generally, PD is diagnosed by a neurological history and clinical exam for the cardinal symptoms of Parkinson's disease (resting tremor, bradykinesa and rigidity). Individuals may also be evaluated for postural instability and unilateral onset. In some instances, a physician may use Unified Parkinson's Disease Rating Scale (UPDRS) or the Movement Disorder Society's revised version of the UPDRS (Goetz et al., Mov Disord. 2007 January; 22(1):41-7). The modified UPDRS uses a four-scale structure with sub scales as follows: (1) non-motor experiences of daily living (13 items), (2) motor experiences of daily living (13 items), (3) motor examination (18 items) and (4) motor complications (6 items). Each subscale now has 0-4 ratings, where 0=normal, 1=slight, 2=mild, 3=moderate, and 4=severe. Clinicians may also use the criteria developed by the U.K. Parkinson's Disease Society Brain bank Clinical Diagnostic Criteria (Hughes A J, Daniel S E, Kilfor L, Lees A J. Accuracy of clinical diagnosis of idiopathic Parkinson's diseases. A clinic-pathological study of 100 cases. JNNP 1992; 55:181-184.)

Huntington's Disease is often defined or characterized by onset of symptoms and progression of decline in motor and neurological function. HD can be broken into five stages: Patients with early HD (stages 1 and 2) have increasing concerns about cognitive issues, and these concerns remain constant during moderate/intermediate HD (stages 3 and 4). Patients with late-stage or advanced HD (stage 5) have a lack of cognitive ability (Ho et al., Clin Genet. September 2011; 80(3):235-239).

Progression of the stages can be observed as follows: Early Stage (stage 1), in which the person is diagnosed as having HD and can function fully both at home and work. Early Intermediate Stage (stage 2), the person remains employable but at a lower capacity and are able to manage their daily affairs with some difficulties. Late Intermediate Stage (stage 3), the person can no longer work and/or manage household responsibilities and need help or supervision to handle daily financial and other daily affairs. Early Advanced Stage patients (stage 4) are no longer independent in daily activities but is still able to live at home supported by their family or professional careers. In the Advanced Stage (stage 5), the person requires complete support in daily activities and professional nursing care is usually needed. Patients with HD usually die about 15 to 20 years after their symptoms first appear.

Indicia of a slower decline in symptoms of Huntington's Disease are measured using change from baseline in one or more of the following parameters: using standardized tests for (i) functional assessment (e.g., UHDRS Total Functional Capacity, LPAS, Independence Scale); (ii) neuropsychological assessment (e.g., UHDRS Cognitive Assessment, Mattis Dementia Rating Scale, Trail Making Test A and B, Figure Cancellation Test, Hopkins Verbal Learning Test, Articulation Speed Test); (iii) psychiatric assessment (UHDRS Behavioral Assessment, Montgomery and Asberg Depression Rating Scale) and (iv) cognitive assessment (e.g., Dementia Outcomes Measurement Suite (DOMS)).

Fenofibrate

Fenofibrate is a fibrate compound, previously used in the treatment of endogenous hyperlipidemias, hypercholesterolemias and hypertriglyceridemias. The preparation of fenofibrate is disclosed in U.S. Pat. No. 4,058,552, the disclosure of which is incorporated herein by reference in its entirety. Fenofibric acid is the active metabolite of fenofibrate. Fenofibrate is not soluble in water, which limits its absorption in the gastrointestinal (GI) tract. Alternative formulations and strategies have been used to overcome this problem. See U.S. Pat. Nos. 4,800,079 and 4,895,726 (micronized fenofibrate); U.S. Pat. No. 6,277,405 (micronized fenofibrate in a tablet or in the form of granules inside a capsule); U.S. Pat. No. 6,074,670 (the immediate release of micronized fenofibrate in a solid state; U.S. Pat. No. 5,880,148 (combination of fenofibrate and vitamin E); U.S. Pat. No. 5,827,536 (diethylene glycol monoethyl ether (DGME) as solubilizer for fenofibrate); and U.S. Pat. No. 5,545,628 (the combination of fenofibrate with one or more polyglycolyzed glycerides), all of which are incorporated herein in their entireties by this reference. Numerous other derivatives, analogs and formulations are known to one of skill in the art. For example, other esters of p-carbonylphenoxy-isobutyric acids as described in U.S. Pat. No. 4,058,552, which is incorporated herein by reference in its entirety, can be used. Fenofibrate analogs include those defined in U.S. Pat. No. 4,800,079. By way of example, gemfibrozil could be used in the methods disclosed herein.

Fenofibrate is optionally dissolved in a proper solvent or solubilizers. Fenofibrate is known to be soluble in many different solubilizers, including, for example, anionic (e.g. SDS) and non-ionic (e.g. Triton X-100) surfactants, complexing agents (N-methyl pyrrolidone). Liquid and semi-solid formulations with improved bioavailability for oral administration of fenofibrate or fenofibrate derivatives are described in International Patent Application Publication No. WO 2004/002458, which is incorporated herein by reference in its entirety.

Kaempferol

Kaempferol (3,5,7-trihydroxy-2-(4-hydroxyphenyl)-4H-1-benzopyran-4-one), a naturally occurring flavonoid found in many edible plants (e.g., tea, broccoli, cabbage, kale, beans, endive, leek, tomato, strawberries and grapes) and possesses a range of pharmacological features, including antioxidant, anti-inflammatory, neuroprotective, anti-atherogenic, and anticancer properties [19, 20].

Evidence from in vitro and in vivo investigations suggests that kaempferol might provide potential as a therapeutic candidate for Alzheimer's disease (AD). Kaempferol prevents β-amyloid-induced toxicity and aggregation effects in vitro within mouse cortical neurons, PC12 neuroblastoma and T47D human breast cancer cells [21-23]. Likewise, a flavonol mixture from Ginkgo leaves, containing quercetin, kaempferol and isorhamnetin, stimulated the BDNF signaling pathway and reduced β-amyloid accumulation within neurons isolated from a double transgenic AD mouse model (TgAPPswe/PS1e9). In vivo studies in these double transgenic AD mice confirmed enhanced BDNF expression following flavonol administration, correlating with improved cognitive function [24]. Kaempferol was also noted to inhibit oxidative stress, elevate superoxide dismutase (SOD) activity in the hippocampus, and improve learning and memory capabilities in mice with D-galactose-induced memory impairment [25]. Pre-treatment with kaempferol or products containing kaempferol provide protection against dopaminergic neurotoxicity within MPTP, 6-OHDA, or rotenone neurotoxicant animal models of PD [26-29].

Pharmaceutical Compositions and Routes of Administration

In some embodiments, the fenofibrate or analog thereof and kaempferol are formulated into one or more compositions with a suitable carrier, excipient or diluent. In some embodiments, the fenofibrate or analog thereof and kaempferol are formulated into the same composition. In alternative embodiments, the fenofibrate or analog thereof and kaempferol are formulated into separate compositions. In some embodiments, the fenofibrate or analog thereof and kaempferol are administered concomitantly (optionally in the same or different compositions). In some embodiments, the fenofibrate or analog thereof and kaempferol are administered sequentially.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Carrier encompasses any excipient, diluent, filler, salt, buffer, stabilizer, solubilizer, lipid, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Depending on the intended mode of administration, the pharmaceutical composition can be in the form of solid, semi-solid, or liquid dosage forms, such as, for example, tablets, suppositories, pills, capsules, powders, liquids, aerosols, or suspensions, preferably in unit dosage form suitable for single administration of a precise dosage. The compositions will include a therapeutically effective amount of the compound(s) described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained. Compositions containing fenofibrate or analog thereof and/or kaempferol described herein or pharmaceutically acceptable salts or prodrugs thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Compositions described herein can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or pharmaceutically acceptable salts or prodrugs thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of fenofibrate or analog thereof and kaempferol or pharmaceutically acceptable salts or prodrugs thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or pharmaceutically acceptable salts or prodrugs thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Fenofibrate or analog thereof and kaempferol can be administered to a neural cell or neural progenitor cell in any number of ways, including, for example, ex vivo, in vitro, and in vivo. In vivo administration can be directed to central or peripheral nervous system neural cells. Thus, in vivo contact can be useful if the subject has or is at risk of developing reduced PGC-1α levels in the central nervous system. In some embodiments, the fenofibrate and kaempferol is administered by intracerebroventricular (ICV) administration.

In vitro contact can be desired for example in treating cells for transplantation. The neural cells can be explants from the nervous system of the same or different subject, can be derived from stem cells, or can be derived from a cell line. The neural cells can be derived from a non-neural cell that is de-differentiated and then caused to differentiate into a neural cell lineage. Such a cell can be an induced pluripotent stem cell. Because fenofibrate crosses the blood brain barrier, a neural cell in the central nervous system can be contacted with the fenofibrate by a systemic administration of the fenofibrate to the subject. The fenofibrate can be administered intrathecally, for example, by local injection, by a pump, or by a slow release implant.

The customary adult fenofibrate dosage is three gelatin capsules per day, each containing 100 mg of fenofibrate. One of skill in the art can select a dosage or dosing regimen by selecting an effective amount of the fenofibrate. Such an effective amount includes an amount that induces PGC-1α expression in neural cells, an amount that has anti-inflammatory properties, an amount that reduces one or more effects of oxidative stress. Additionally, the effective amount of fenofibrate increases levels of phosphorylated AMPK, increases mitochondrial number, and increases cell viability. It is contemplated that administration of fenofibrate or analog thereof and kaempferol in combination will reduce the effective dose of fenofibrate or analog thereof necessary in a subject compared to administration of fenofibrate or analog thereof alone.

Optionally, the fenofibrate or analog thereof and kaempferol is administered daily.

The term “effective amount”, as used herein, is defined as any amount sufficient to produce a desired physiologic response. By way of example, the systemic dosage of the fenofibrate or analog thereof and kemopferol can be 1-1000 mg daily, including for example, 300 to 400 mg daily (administered for example in 1-5 doses). One of skill in the art would adjust the dosage as described below based on specific characteristics of the inhibitor, the subject receiving it, the mode of administration, type and severity of the disease to be treated or prevented, and the like. Furthermore, the duration of treatment can be for days, weeks, months, years, or for the life span of the subject. For example, administration to a subject with or at risk of developing a neurodegenerative disease could be at least daily (e.g., once, twice, three times per day), every other day, twice per week, weekly, every two weeks, every three weeks, every 4 weeks, every 6 weeks, every 2 months, every 3 months, or every 6 months, for weeks, months, or years so long as the effect is sustained and side effects are manageable.

Effective amounts and schedules for administering fenofibrate or analog thereof and kaempferol can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, cell death, and the like. Generally, the dosage will vary with the type of neurodegenerative disease, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily.

Combination Therapy

In some embodiments, the methods described herein further comprise administering a standard of care therapeutic for the treatment of a neurodegenerative disease. As used herein, the term “standard of care” refers to a treatment that is generally accepted by clinicians for a certain type of patient diagnosed with a type of illness. In some embodiments, the standard of care therapeutic is levodopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant or tetrabenazine.

In some embodiments, the combination therapy employing fenofibrate or analog thereof and kaempferol described herein may precede or follow administration of additional standard of care therapeutic(s) by intervals ranging from minutes to weeks to months. For example, separate modalities are administered within about 24 hours of each other, e.g., within about 6-12 hours of each other, or within about 1-2 hours of each other, or within about 10-30 minutes of each other. In some situations, it may be desirable to extend the time period for treatment significantly, where several days (2, 3, 4, 5, 6 or 7 days) to several weeks (1, 2, 3, 4, 5, 6, 7 or 8 weeks) lapse between the respective administrations of different modalities. Repeated treatments with one or both agents/therapies of the combination therapy is specifically contemplated.

Monitoring Efficacy of Therapy

Methods for measuring PGC-1α induction and activity are known in the art and are provided in Example 1 below. See, for example, Ruiz et al. (2012) A cardiac-specific robotized cellular assay identified families of human ligands as inducers of PGC-1α expression and mitochondrial biogenesis PLoS One: 7: e46753. PGC-1α levels can be assessed directly using, for example, an antibody to PGC-1α or other means of detection. PGC-1α activity can be detected including by way of example by assessing modulation of mitochondrial function, e.g., oxidative metabolism and can be assessed by detecting the activity or expression of a mitochondrial gene, e.g., LDH-2, ATP5j, or the like.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

### Example 1—Fenofibrate Inhibits Lipopolysaccharide (LPS)-Induced Inflammation in Primary Astrocytes Derived from PGC-1α WT (PGC-1α^{+/+}) and Heterozygous PGC-1α Knockout (PGC-1α^{+/-}) Mice

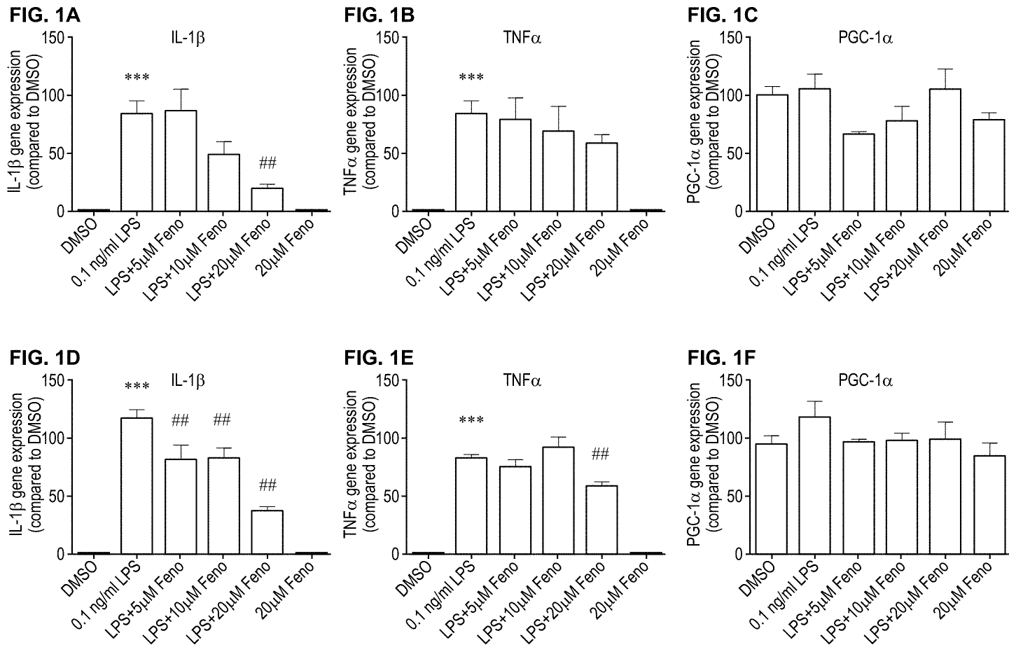

Primary astrocytes from postnatal heterozygous mice were isolated and cultured and wild type mice were obtained by breeding these heterozygous knockout mice. The astrocytes were treated with fenofibrate at various concentration overnight followed by 0.1 ng/mL LPS for 1 hour. Total RNA was isolated, and gene expression of pro-inflammatory cytokines, IL-1β and TNF-α, was determined by RT-PCR. The results show that fenofibrate exerted anti-inflammatory protection effects in both WT (PGC-1a+/+) and heterozygous (PGC-1a+/−) primary astrocytes (FIG. 1). These data suggest that while fenofibrate is active in both types of astrocytes, it is more active in astrocytes carrying a single copy of the Ppargc1a. This may also have implications for neurodegenerative diseases such as Alzheimer's Disease (AD), Parkinson's Disease (PD) and amyotrophic lateral sclerosis (ALS) where PGC-1α levels are pathologically reduced.

### Example 2—PPARα is not Required for Fenofibrate-Mediated Anti-Inflammatory Effects in Mouse Primary Astrocytes

The following Example demonstrates that fenofibrate-mediated anti-inflammatory effects were not suppressed in mouse primary astrocytes after silencing of PPARα expression by siRNA.

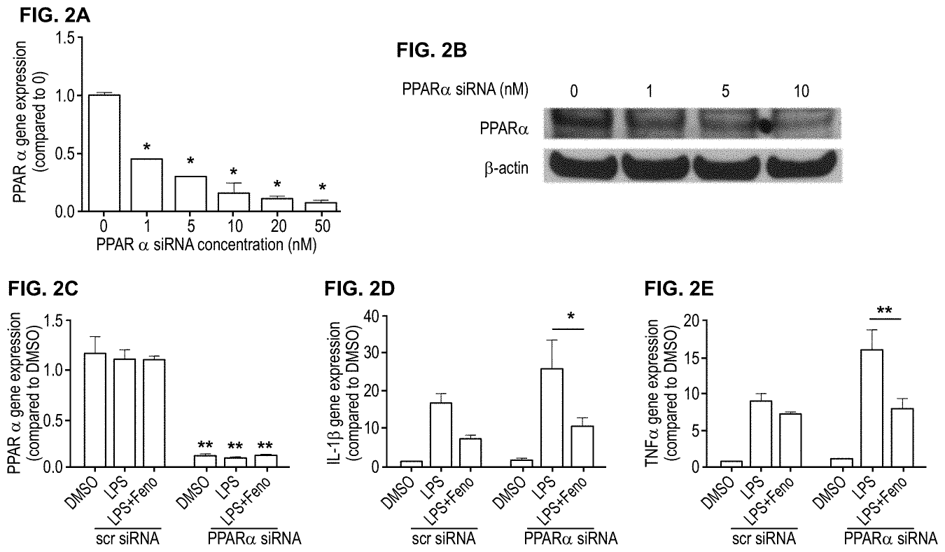

Different concentrations of PPARα siRNA were added to mouse primary astrocytes for 48 hrs. Total RNA and protein was collected. PPARα gene expression was determined by qRT-PCR (FIG. 2A) and protein expression was determined by western blot analysis (FIG. 2B). Then 10 nM PPARα siRNA was used for the subsequent experiments. (FIGS. 2C-2E) Primary astrocytes were treated with 10 nM PPARα siRNA or scrambled siRNA for 30 hours followed by 20 μM fenofibrate for another 18 hrs. Then the cells were treated with 0.1 ng/ml LPS for 1 hr. Total RNA was extracted for PPARα (FIG. 2C), IL-1β (FIG. 2D), TNFα (FIG. 2E) gene expression. The results indicate that fenofibrate mediated anti-inflammatory effects in a PPARα-independent manner in the teo major neuroglial cell populations.

### Example 3—Fenofibrate Undergoes Rapid First-Pass Hydrolysis to Fenofibric Acid In Vivo

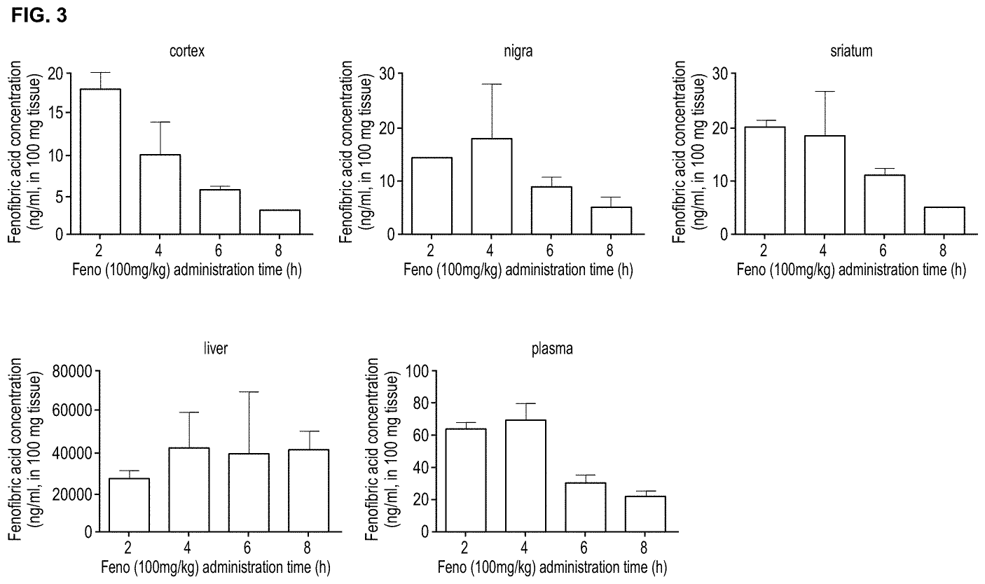

It is reported that after oral administration fenofibrate is rapidly converted to fenofibric acid, the active metabolite and PPARα ligand involved in promoting the anti-hyperlipidemic activity [17, 18]. The pharmacokinetics of fenofibrate was measured in brain, liver and plasma of the mice that received an oral dose of 100 mg/kg of fenofibrate. The majority of fenofibrate was metabolized in the liver to fenofibric acid; with only a small portion of the fenofibric acid entering the bloodstream and the brain (FIG. 3).

### Example 4—Anti-Inflammatory Properties are Mediated by Fenofibrate and not Fenofibric Acid, its First-Pass Metabolite, in BV2 Cells

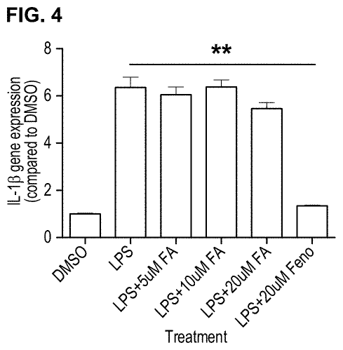

Fenofibrate is rapidly hydrolyzed in vivo during a first-pass through the liver, metabolized by carboxylesterase enzymes to fenofibric acid. Fenofibric acid is reported to be the active moiety that provides lipid-lowering properties of oral fenofibrate. Whether the neuroprotective properties of fenofibrate are dependent on the parent molecule or its primary metabolite had not been previously defined. This has been a major disadvantage in the current pursuit of fenofibrate therapy as treatment for neurodegenerative diseases. As the prodrug fenofibrate has been used for all previous in vitro assays, whether fenofibric acid can equally exert anti-inflammatory effect was also assessed. To test this, BV2 cells were treated with either fenofibric acid (FA) at different concentrations (0, 5, 10, and 20 μM) or 20 μM fenofibrate for 18 hours, followed by a one-hour LPS exposure. Total BV2 cell RNA was extracted for IL-1β gene expression via qRT-PCR analysis. Surprisingly, it was discovered that fenofibric acid did not inhibit IL-1β expression at any concentration, while 20 μM fenofibrate exerts a robust anti-inflammatory effect (FIG. 4). These results revealed that fenofibrate, and not fenofibric acid, mediated the anti-inflammatory effects seen in previous experiments.

### Example 5—Kaempferol Prevents the Hydrolysis of Fenofibrate to Fenofibric Acid Via Inhibition of Carboxylesterase Esterase (hCES1b) In Vitro

The potential of kaempferol as a naturally occurring esterase inhibitor was explored, effective in reducing fenofibrate hydrolysis to fenofibric acid in the liver. Human carboxylesterases (CESs) belong to the serine esterase super family and are classified into five CES (1-5) groups. The CES1 and CES2 sub-families are the most important participants in the hydrolysis of a variety of xenobiotics and drugs in humans. Human CES1 is highly expressed within the liver and contributes predominantly to the intrinsic hydrolase/esterase activities. The human CES1 isoform is also found at low levels in the small intestine, macrophages, lung epithelia, heart and testis. The human CES1A is further classified into two isoforms: hCES1b (also referred to as CES1A1) and hCES1c. Studies suggest that hCES1b is the major (wild-type) isoform functioning within human liver, important for the hydrolysis of substrates containing ester/thioester/amide bonds, including fenofibrate. Hence, in a series of studies the potency of kaempferol to specifically inhibit recombinant human CES1b-mediated ability hydrolysis of fenofibrate to fenofibric acid was studied using an enzyme inhibition assay. Next, the overall esterase inhibiting property of kaempferol on other liver esterases using pooled human liver microsomes (HLM) was assessed.

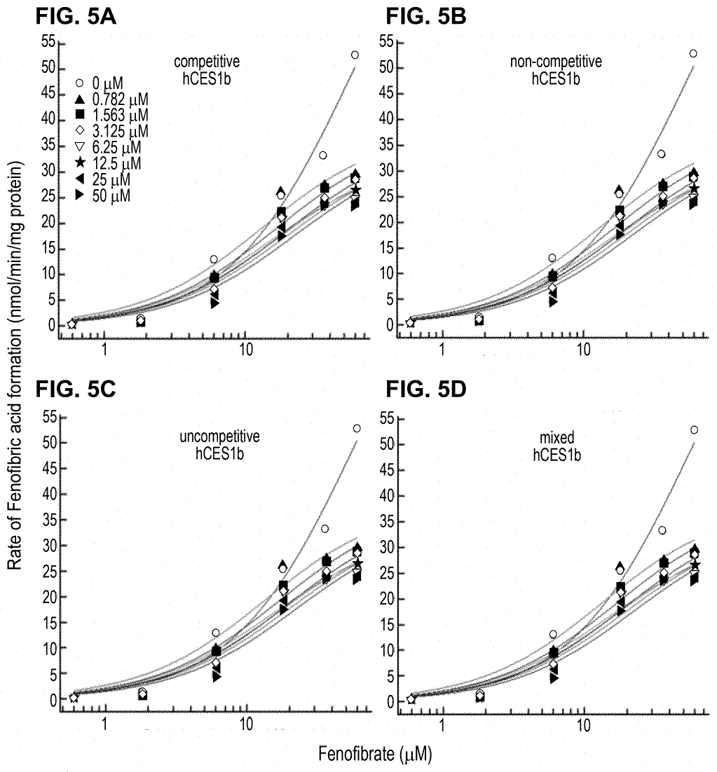

Determination of Km and Vmax values: First, the Michaelis-Menten constant (Km), the substrate concentration at which half maximum velocity is observed, and Vmax (the maximum rate of the reaction) values were determined for the hydrolysis of fenofibrate to fenofibric acid using either hCES1b or HLM in the following enzyme assay. Assay procedure: Incubation mixtures containing 100 mM Tris-Cl buffer (pH 7.4), and recombinant hCES1b (0.05 mg/mL) or pooled HLM (1 mg/mL) were warmed to 37° C. Different concentrations of fenofibrate was added to start the 10-minute assay. The reactions were stopped by the addition of a stop solution containing an internal standard. Samples were centrifuged to precipitate the protein while the supernatant was collected for determination of fenofibric acid using liquid chromatography-tandem mass spectrometry (LC-MS/MS) analysis. Standard curves for the hydrolysis of fenofibrate to fenofibric acid by either recombinant hCES1b (FIG. 5A) or HLM (FIG. 5B) were plotted. The Km and Vmax values were calculated by fitting data to enzyme kinetics models (Table 1).

TABLE 1
Km and Vmax values for the hydrolysis of fenofibrate to fenofibric
acid using the matrix recombinant hCES1b and HLM.

| Compound | Product | Matrix | Kₘ (μM) | Vₘₐₓ (nmol/min/mg) |
|---|---|---|---|---|
| Fenofibrate | Fenofibric acid | hCES1b | 6.04 | 28.3 |
| | | HLM | 5.40 | 96.3 |

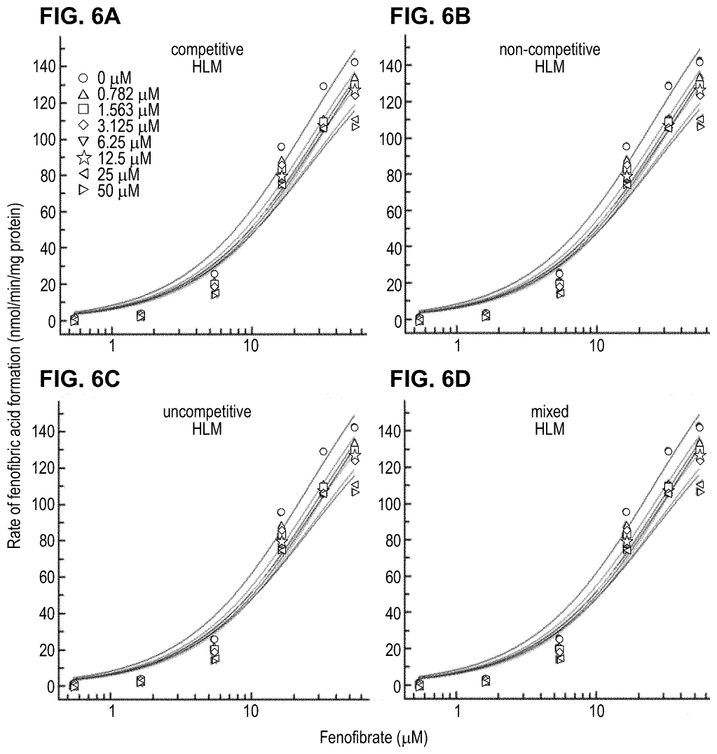

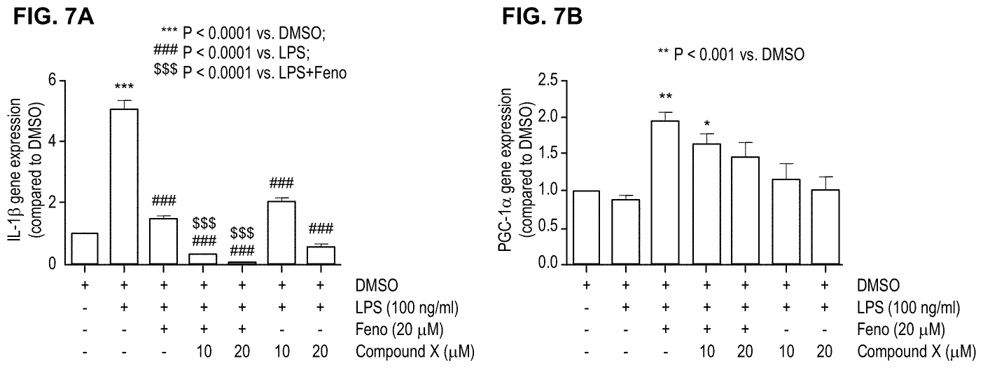

Determination of inhibition constant (Ki) for kaempferol: Next, the inhibition constant (Ki, the concentration required to produce half maximum inhibition) of kaempferol in preventing the hydrolysis of fenofibrate to fenofibric acid by either hCES1b (FIG. 6A-6D) or HLM (FIG. 7A-7D) was determined. Incubation mixtures containing 100 mM Tris-Cl buffer (pH 7.4), recombinant hCES1b (0.05 mg/mL) or pooled human liver microsomes (1 mg/mL) and 8 concentrations of kaempferol or a positive control inhibitor (bis(4-nitrophenyl)-phosphate, BNP) were pre-incubated for 2 minutes at 37° C. Fenofibrate was then added to start the 10-minute reaction (final concentrations: 0.1×Km, 0.3×Km, 1×Km, 3×Km, 6×Km, and 10×Km). The reactions were stopped by the addition of a stop solution containing an internal standard. Samples were centrifuged to precipitate protein and the supernatant was collected for LC-MS/MS analysis. The fenofibric acid was quantified using standard curves. The Ki values were calculated, and the type of inhibition was determined by fitting data to specific enzyme inhibition models (Table 2). Kaempferol specifically inhibited the hCES1b hydrolase activity, with a low Ki value of 36.2 μM, while inhibiting the pooled HLM with a higher Ki value of 110 μM, calculated using a competitive inhibition model.

TABLE 2
Ki values of kaempferol for inhibiting the hydrolysis of fenofibrate to fenofibric acid using
the matrix recombinant hCES1b and HLM. BNP-bis(4-nitrophenyl)-phosphate - positive control.

| | | Inhibition Model | | | | | | | |
|---|---|---|---|---|---|---|---|---|---|
| Test | | Competitive | | Non-Competitive | | Uncompetitive | | Mixed | |
| Compound | Matrix | Kᵢ (μM) | R² | Kᵢ (μM) | R² | Kᵢ (μM) | R² | Kᵢ (μM) | R² |
| Kaempferol | hCES1b | 36.2 | 0.900 | 101 | 0.903 | 64.7 | 0.902 | 101 | 0.903 |
| BNP | | 0.044 | 0.920 | 0.170 | 0.917 | 0.096 | 0.882 | 0.044 | 0.920 |
| Kaempferol | HLM | 110 | 0.974 | 235 | 0.975 | 131 | 0.974 | 110 | 0.974 |
| BNP | | 1.67 | 0.828 | 5.17 | 0.826 | 3.02 | 0.818 | 1.66 | 0.828 |

These above findings suggest, therefore, that kaempferol specifically inhibits the hydrolase activity of hCES1b, an important enzyme involved in the hydrolysis of fenofibrate in the human liver. Thus, kaempferol is a potential candidate for use in combination with fenofibrate, to inhibit the first-pass metabolism of fenofibrate to fenofibric acid and thereby enhance fenofibrate's potential for CNS bioavailability.

### Example 6—Co-Delivery of Fenofibrate and Kaempferol Exert Synergistic Anti-Inflammatory Effects in BV2 Cells

Given that the anti-inflammatory properties appear to be mediated by the prodrug, fenofibrate, and not its active metabolite fenofibric acid, it was attempted to increase PGC-1α expression within the CNS by enhancing fenofibrate's bioavailability. It was contemplated that enhancing fenofibrate levels in the CNS would lead to a more robust PGC-1α-mediated neuroprotective effect. The following Example provides a method to increase CNS fenofibrate levels by inhibiting the first-pass hydrolysis of fenofibrate to fenofibric acid by carboxylesterase in the liver.

The anti-inflammatory effect of co-delivery of fenofibrate with kaempferol in BV2 cells was assessed. 20 μM of fenofibrate and/or 10 or 20 μM of kaempferol were added to BV2 cells for 18 hours followed by 1-hour exposure to LPS. Cell lysates were collected for determination of IL-1β and PGC-1α gene expression via qRT-PCR. Kaempferol treatment alone inhibited IL-1β expression (FIG. 7A), supportive of reported anti-inflammatory properties [20]. Co-delivery of fenofibrate and kaempferol exerted an additive, if not synergistic anti-inflammatory effect (FIG. 7A), suggesting combination therapy might be efficacious in diseases featuring underlying levels of neuroinflammation. It seemed, however, that kaempferol slightly repressed fenofibrate-mediated PGC-1α gene up-regulation when the former was delivered at high doses (FIG. 7B).

### Example 7— Co-Delivery of Fenofibrate and Kaempferol Increased Brain Fenofibrate Levels In Vivo in Mice

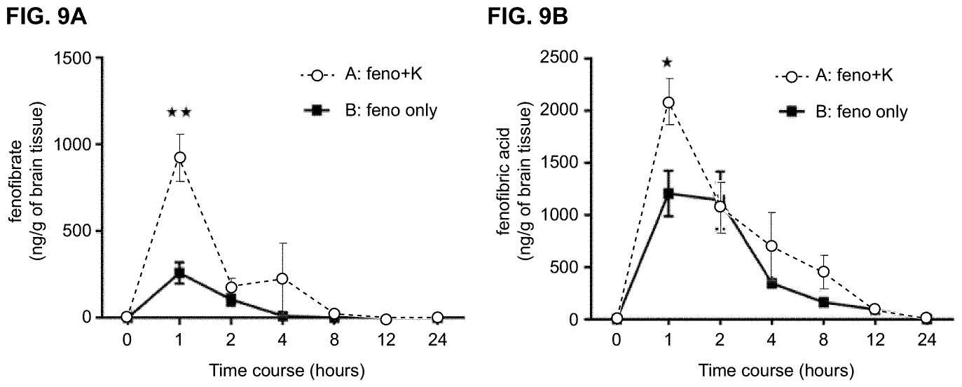

Next, the ability of kaempferol to enhance brain fenofibrate levels in vivo was assessed in naïve C57/BL mice. C57/BL6 mice were divided into two groups: group A (n=28) and group B (n=28). C57/BL6 mice in group A were pre-treated for 2 days with kaempferol (50 mg/kg) and on day 3 received the kaempferol (50 mg/kg) and fenofibrate (100 mg/kg) combination. Group B mice received vehicle for 2 days and on day 3 were administered fenofibrate (100 mg/kg) only. All the drug administrations were performed via oral gavage. Mice were subsequently sacrificed at seven different timepoints following the drug administration(s), at 0, 1, 2, 4, 8, 12 and 24-hours, respectively. Brain tissue was collected, immediately frozen in liquid nitrogen, and stored at −80° C. until analysis. Frozen brain tissue was homogenized in a methanol:water mixture (20:80), centrifuged to precipitate proteins, and the supernatant collected to determine quantitative levels of fenofibrate and fenofibric acid via LC-MS/MS. Co-delivery of kaempferol with fenofibrate increased brain fenofibrate (FIG. 9A) and fenofibric acid (FIG. 9B) levels at the 1-hour timepoint, when their levels appear to peak. The levels of fenofibrate and fenofibric acid were maintained at higher concentrations for at least 4-8 (F and FA, respectively) hours in mice receiving both fenofibrate and kaempferol compared to those receiving fenofibrate only. These murine in vivo results suggest that kaempferol administration can be used to enhance brain fenofibrate levels.

### Example 8— Co-Delivery of Fenofibrate and Kaempferol Potentiated Neuroprotection in MPTP Mouse Model of Parkinson's Disease (PD)

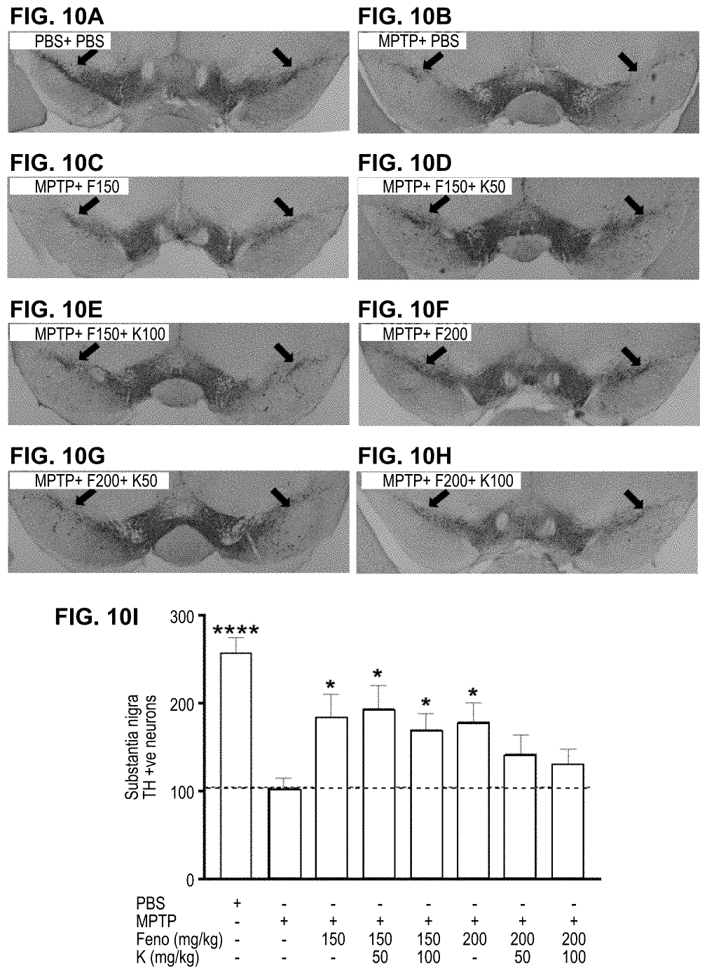

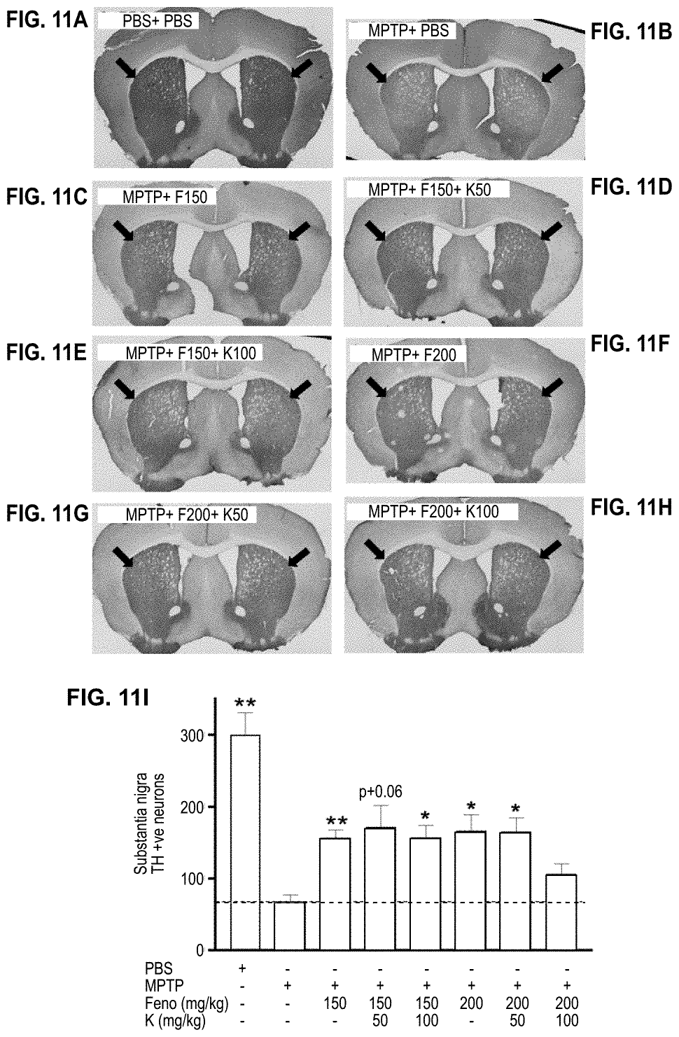

The neuroprotective effects of co-delivery of kaempferol and fenofibrate was studied in a mouse model of PD. At 13 weeks of age, C57/BL6 mice were treated with either MPTP (30 mg/kg) or saline (i.p.) for five consecutive days, followed by either 14 days of i.p. saline or i.p. fenofibrate and/or kaempferol treatment. The C57/BL6 mice were divided into eight groups of 8 animals per group; Group A: saline+saline treatment; Group B: MPTP+saline treatment; Group C: MPTP+150 mg/kg fenofibrate; Group D: MPTP+150 mg/kg fenofibrate and 50 mg/kg kaempferol treatment; Group E: MPTP+150 mg/kg fenofibrate and 100 mg/kg kaempferol treatment; Group F: MPTP+200 mg/kg fenofibrate; Group G: MPTP+200 mg/kg fenofibrate and 50 mg/kg kaempferol treatment; Group H: MPTP+200 mg/kg fenofibrate and 100 mg/kg kaempferol treatment. At the end of treatment these animals were sacrificed, perfused with paraformaldehyde (PFA), and brain sections stained for tyrosine hydroxylase (TH) for immunohistochemical analysis. It was observed that 5-day MPTP (30 mg/kg) sub-chronic treatment induced significant loss of dopaminergic neurons in the substantia nigra (FIG. 10B), with associated neurite loss in striatum (FIG. 11B) when compared to saline treated mice (FIG. 10A, 11A). Subsequent fenofibrate treatment (150 and 200 mg/kg) prevented MPTP-induced loss of nigral neurons (FIG. 10C, 10F) and striatal neurites (FIG. 11C, 11F), confirming our previous findings. Co-administration of fenofibrate (150 mg/kg) with kaempferol (50 mg/kg) increased the neuroprotective effect (FIG. 10D, 11D) compared to treatment with fenofibrate alone, indicating that kaempferol acts additively to prevent MPTP-induced neurotoxicity. The co-administration of a very high dose of fenofibrate, however, together with high dose kaempferol (100 mg/kg) failed to show an improvement in neuroprotection (FIG. 10I, 11I), indicating that these two drugs must be delivered in a fixed mass ratio to elicit maximum neuroprotection.

### Example 9—Green Tea and Capers are Potential Natural Sources of Kaempferol and its Derivatives

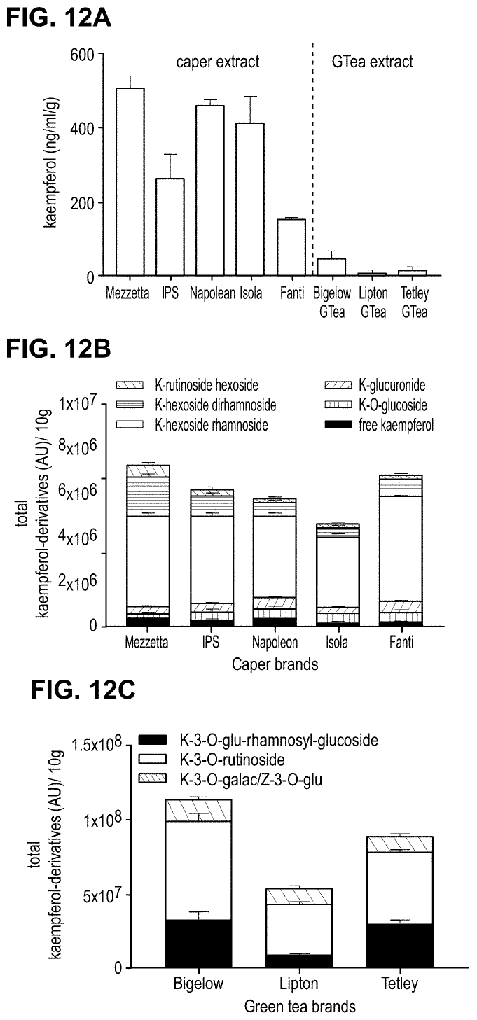

Co-administration of kaempferol with fenofibrate as a neuroprotective therapy to treat patients at risk of or suffering from neurodegenerative disorders and traumatic brain injury is specifically contemplated. Kaempferol has intrinsic activity as an anti-inflammatory and may be separately formulated as a nutraceutical. Hence, the relative amount of kaempferol in natural sources containing the molecule, such as capers and green tea, was investigated using triple quadrupole-MS (TQ-MS) analysis. Five different brands of capers (Mezzetta, IPS, Napoleon, Isola, Fanti) and three brands of green tea (Bigelow, Lipton, Tetley) that were purchased at a local retail store. Capers were extracted with MeOH:water (1:1) for 24 hours at room temperature, while green tea was extracted in boiling water for three minutes. The TQ-MS results showed that higher quantities of ‘free’ kaempferol were present in the caper extract (160-505 ng/ml/g) compared to green tea extract (14-50 ng/ml/g) (FIG. 15A). On the other hand, quad time of flight (QTOF) MS qualitative analysis (per their m/z) of the two extracts showed 1-2 orders of magnitude higher levels of kaempferol-derivatives contained in the green tea extract (5×10⁷-1.2×10⁸ AU) compared to caper extract (4×10⁶-7×10⁶AU) (FIG. 12B, 12C). TQ-MS only provides the amount of ‘free’ kaempferol and not the contained kaempferol-derivatives (conjugated with complex molecules), with the latter determined by QTOF MS qualitative analysis. Overall, the results suggest that green tea extract contains high amounts of kaempferol-derivatives that might provide an alternative source for that molecule.

Publications cited herein and the materials for which they are cited are hereby specifically incorporated by reference in their entireties. A number of embodiments have been described. Nevertheless, it will be understood that various modifications may be made. Accordingly, other embodiments are within the scope of the following claims.

### REFERENCES

1. Agarwal, S., A. Yadav, and R. K. Chaturvedi, Peroxisome proliferator-activated receptors (PPARs) as therapeutic target in neurodegenerative disorders. Biochem Biophys Res Commun, 2016.
2. Carta, A. R., et al., Rosiglitazone decreases peroxisome proliferator receptor-gamma levels in microglia and inhibits TNF-alpha production: new evidences on neuroprotection in a progressive Parkinson's disease model. Neuroscience, 2011. 194: p. 250-61.
3. Combs, C. K., et al, Inflammatory mechanisms in Alzheimer's disease: inhibition of beta-amyloid-stimulated proinflammatory responses and neurotoxicity by PPARgamma agonists. J Neurosci, 2000. 20(2): p. 558-67.
4. Mandrekar-Colucci, S., J. C. Karlo, and G. E. Landreth, Mechanisms underlying the rapid peroxisome proliferator-activated receptor-gamma-mediated amyloid clearance and reversal of cognitive deficits in a murine model of Alzheimer's disease. J Neurosci, 2012. 32(30): p. 10117-28.
5. Mandrekar-Colucci, S., et al., PPAR agonists as therapeutics for CNS trauma and neurological diseases. ASN Neuro, 2013. 5(5): p. e00129.
6. Pisanu, A., et al., Dynamic changes in pro- and anti-inflammatory cytokines in microglia after PPAR-gamma agonist neuroprotective treatment in the MPTPp mouse model of progressive Parkinson's disease. Neurobiol Dis, 2014. 71: p. 280-91.
7. Wu, Z., et al., Mechanisms controlling mitochondrial biogenesis and respiration through the thermogenic coactivator PGC-1. Cell, 1999. 98(1): p. 115-24.
8. Eschbach, J., et al., PGC-1alpha is a male-specific disease modifier of human and experimental amyotrophic lateral sclerosis. Hum Mol Genet, 2013. 22(17): p. 3477-84.
9. Qin, W., et al., PGC-1alpha expression decreases in the Alzheimer disease brain as a function of dementia. Arch Neurol, 2009. 66(3): p. 352-61.
10. Zheng, B., et al., PGC-1alpha, a potential therapeutic target for early intervention in Parkinson's disease. Sci Transl Med, 2010. 2(52): p. 52ra73.
11. Ventura-Clapier, R., A. Gamier, and V. Veksler, Transcriptional control of mitochondrial biogenesis: the central role of PGC-1alpha. Cardiovasc Res, 2008. 79(2): p. 208-17.
12. Chanda, D., et al., Fenofibrate differentially regulates plasminogen activator inhibitor-1 gene expression via adenosine monophosphate-activated protein kinase-dependent induction of orphan nuclear receptor small heterodimer partner. Hepatology, 2009. 50(3): p. 880-92.
13. Steinberg, G. R. and B. E. Kemp, AMPK in Health and Disease. Physiological reviews, 2009. 89(3): p. 1025-78.
14. Jager, S., et al., AMP-activated protein kinase (AMPK) action in skeletal muscle via direct phosphorylation of PGC-1alpha. Proceedings of the National Academy of Sciences of the United States of America, 2007. 104(29): p. 12017-22.
15. Handschin, C., et al., An autoregulatory loop controls peroxisome proliferator-activated receptor gamma coactivator 1alpha expression in muscle. Proceedings of the National Academy of Sciences of the United States of America, 2003. 100(12): p. 7111-6.
16. Chen, L. W., et al., Activating mitochondrial regulator PGC-1alpha expression by astrocytic NGF is a therapeutic strategy for Huntington's disease. Neuropharmacology, 2012. 63(4): p. 719-32.
17. Caldwell, J., The biochemical pharmacology of fenofibrate. Cardiology, 1989. 76 Suppl 1: p. 33-41; discussion 41-4.
18. Godfrey, A. R., J. Digiacinto, and M. W. Davis, Single-dose bioequivalence of 105-mg fenofibric acid tablets versus 145-mg fenofibrate tablets under fasting and fed conditions: a report of two phase I, open-label, single-dose, randomized, crossover clinical trials. Clin Ther, 2011. 33(6): p. 766-75.
19. Calderon-Montano, J. M., et al., A review on the dietary flavonoid kaempferol. Mini Rev Med Chem, 2011. 11(4): p. 298-344.
20. Chen, A. Y. and Y. C. Chen, A review of the dietary flavonoid, kaempferol on human health and cancer chemoprevention. Food Chem, 2013. 138(4): p. 2099-107.
21. Roth, A., W. Schaffner, and C. Hertel, Phytoestrogen kaempferol (3,4′,5,7-tetrahydroxyflavone) protects PC12 and T47D cells from beta-amyloid-induced toxicity. J Neurosci Res, 1999. 57(3): p. 399-404.
22. Ono, K., et al., Potent anti-amyloidogenic and fibril-destabilizing effects of polyphenols in vitro: implications for the prevention and therapeutics of Alzheimer's disease. J Neurochem, 2003. 87(1): p. 172-81.
23. Choi, S. M., et al., Effects of Flavonoid Compounds on beta-amyloid-peptide-induced Neuronal Death in Cultured Mouse Cortical Neurons. Chonnam Med J, 2014. 50(2): p. 45-51.
24. Hou, Y., et al., Anti-depressant natural flavonols modulate BDNF and beta amyloid in neurons and hippocampus of double TgAD mice. Neuropharmacology, 2010. 58(6): p. 911-20.
25. Lei, Y., et al., In vivo investigation on the potential of galangin, kaempferol and myricetin for protection of D-galactose-induced cognitive impairment. Food Chem, 2012. 135(4): p. 2702-7.
26. Ablat, N., et al., Neuroprotective Effects of a Standardized Flavonoid Extract from Safflower against a Rotenone-Induced Rat Model of Parkinson's Disease. Molecules, 2016. 21(9).
27. Ren, R., et al., Neuroprotective Effects of A Standardized Flavonoid Extract of Safflower Against Neurotoxin-Induced Cellular and Animal Models of Parkinson's Disease. Sci Rep, 2016. 6: p. 22135.
28. Li, S. and X. P. Pu, Neuroprotective effect of kaempferol against a 1-methyl-4-phenyl-1,2,3,6-tetrahydropyridine-induced mouse model of Parkinson's disease. Biol Pharm Bull, 2011. 34(8): p. 1291-6.
29. Datla, K. P., et al., The antioxidant drink effective microorganism-X (EM-X) pre-treatment attenuates the loss of nigrostriatal dopaminergic neurons in 6-hydroxydopamine-lesion rat model of Parkinson's disease. J Pharm Pharmacol, 2004. 56(5): p. 649-54.

1. A method for treating a neurodegenerative disease in a subject comprising administering fenofibrate and kaempferol to a subject in need thereof.

2. (canceled)

3. The method of claim 1, wherein the subject
(a) is at risk for developing a neurodegenerative disease; or
(b) has an early stage neurodegenerative disease.

4. (canceled)

5. The method of claim 1, wherein
the neurodegenerative disease is Parkinson's Disease, Parkinson-plus syndrome, familial dementia, vascular dementia, Alzheimer's Disease, Huntington's Disease, multiple sclerosis, dementia with Lewy bodies, Mild Cognitive Impairment, frontotemporal dementia, retinal neurodegeneration, Amyotrophic Lateral Sclerosis (ALS) or traumatic brain injury (TBI).

6. The method of claim 5, wherein the Parkinson-plus syndrome is selected from the group consisting of multiple system atrophy (MSA), progressive supranuclear palsy (PSP), and corticobasal degeneration (CBD).

7. The method of claim 1, wherein the fenofibrate and kaempferol are administered concomitantly.

8. The method of claim 1, wherein the fenofibrate and kaempferol are administered sequentially.

9. The method of claim 1, further comprising administering a standard of care therapeutic to the subject, wherein the standard of care therapeutic is a dopamine precursor, dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, or tetrabenazine and derivatives thereof.

10-11. (canceled)

12. The method of claim 1, wherein the fenofibrate and kaempferol are administered at a fixed molar ratio.

13. The method of claim 12, wherein the molar ratio of fenofibrate to kaempferol is 1.5:1, 2:1, 3:1, or 4:1.

14. The method of claim 1, wherein the administration increases levels of fenofibrate in the brain compared to treatment with fenofibrate alone.

15. The method of claim 14, wherein the levels are increased for at least 2-4 hours.

16-17. (canceled)

18. A method to prevent/reduce the first-pass metabolism of fenofibrate to fenofibric acid and thereby augment levels of fenofibrate in a subject comprising administering a combination of fenofibrate and kaempferol in a molar ratio sufficient for reducing first pass metabolism of fenofibrate.

19. The method of claim 18, wherein the subject has a neurodegenerative disease.

20. The method of claim 18, wherein the neurodegenerative disease is Parkinson's Disease, Parkinson-plus syndrome, familial dementia, vascular dementia, Alzheimer's Disease, Huntington's Disease, multiple sclerosis, dementia with Lewy bodies, Mild Cognitive Impairment, frontotemporal dementia, retinal neurodegeneration, Amyotrophic Lateral Sclerosis (ALS) or traumatic brain injury.

21. The method of claim 20, wherein the Parkinson-plus syndrome is multiple system atrophy (MSA), progressive supranuclear palsy (PSP) or corticobasal degeneration (CBD).

22. The method of claim 18, further comprising administering a standard of care therapeutic to the subject, wherein the standard of care therapeutic is a dopamine precursor, dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, or tetrabenazine and derivatives thereof.

23. (canceled)

24. A method of inducing PGC-1α expression in a neural cell or a neural progenitor cell comprising contacting a neural cell or a neural progenitor cell with fenofibrate and kaempferol.

25-26. (canceled)

27. The method of claim 24 wherein the neural cell is a neuron.

28. The method of claim 27, wherein the neuron is
(a) a dopaminergic neuron; or
(b) neuron is from a cortex, striatum or spinal cord of a subject.

29. (canceled)

30. The method of claim 24, wherein the neural cell is a glial cell or astrocyte.

31-32. (canceled)

33. The method of claim 1, wherein the kaempferol is from a plant or plant extract comprising kaempferol.

34. The method of claim 33, wherein the plant or plant extract is from green tea, capers, kale, tea, broccoli, cabbage, beans, endive, leek, tomato, strawberries or grapes.
